(19) United States
(12) Patent Application Publication
(10) Pub. No.: US 2025/0051285 A1
(43) Pub. Date: Feb. 13, 2025

(54) CRYSTALLINE SALT FORM OF EP4 ANTAGONIST

(71) Applicant: Eisai R&D Management Co., Ltd., Bunkyo-ku, Tokyo (JP)

(72) Inventor: Farid Benayoud, North Andover, MA (US)

(73) Assignee: Eisai R&D Management Co., Ltd., Bunkyo-ku, Tokyo (JP)

(21) Appl. No.: 18/719,032

(22) PCT Filed: Dec. 22, 2022
(86) PCT No.: PCT/US2022/053842

§ 371 Date: Jun. 12, 2024

Related U.S. Application Data
(60) Provisional application No. 63/293,183, filed on Dec. 23, 2021.

(51) Int. Cl. C07D 231/20; A61K 31/415

(52) U.S. Cl. CPC C07D 231/20; A61K 31/415

(57) ABSTRACT

A crystalline free acid form of (S)-4-(1-(3-(difluoromethyl)-1-methyl-5-(3-(trifluoromethyl)phenoxy)-1H-pyrazole-4-carboxamido)ethyl)benzoic acid is provided. Methods of making and using the crystalline free acid form of (S)-4-(1-(3-(difluoromethyl)-1-methyl-5-(3-(trifluoromethyl)phenoxy)-1H-pyrazole-4-carboxamido)ethyl)benzoic acid are also provided.

### CROSS-REFERENCE TO RELATED APPLICATIONS

This application claims the benefit of U.S. Provisional Patent Application No. 63/293,183, filed on Dec. 23, 2021. That application is incorporated by reference as if fully rewritten herein.

### BACKGROUND

Blocking of prostaglandin E2 (PGE2) signaling through the interaction of PGE2 with the prostaglandin E receptor 4 (EP4) by antagonists has been shown to be effective in reducing inflammation (Chen et al. (2010) British J. Pharmacol. 160, 292-310). PGE2 has also been implicated as an important constituent in the immunosuppressive environment created by many solid tumors (Whiteside (2010) Expert Opinion in Biological Therapy. 2010.10, 1019-1035), and inhibition of EP4 signaling by antagonists were shown to reduce tumor growth (Terada et al. (2010) Cancer Res. 70, 1606-1615) and tumor metastasis in tumor animal models (Yang et al. (2006) Cancer Res. 66, 9665-9672). Further reports of PGE2 type EP4 antagonism used in the oncology field may be found at Ma X. et al. (2013) Oncoimmunology 2:e22647; and Cherukuri, D. P. et al. (2007) Exp. Cell Res. 313, 2969-2979; Majumder, M. et al. (2018) Int. J. Mol. Sci. 19, 1019; and Terada, N. et al. (2010) Cancer Research 70, 1606-1615.

Specific examples of EP4 antagonists include compounds of the chemical structure according to Formula I and are described in U.S. Pat. No. 8,686,018, which is hereby fully incorporated

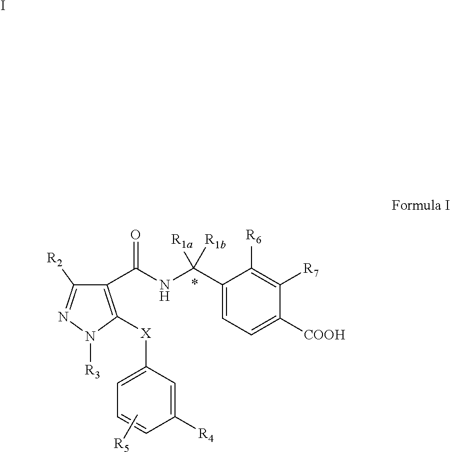

Though the utility of EP4 antagonism has been demonstrated within the art, their manufacture and use as active pharmaceutical ingredients in drug products should be in a form that can be conveniently manipulated and processed. In this regard, chemical stability and physical stability of the active compound are important considerations. Preferably, the compound and pharmaceutical compositions containing it are capable of being effectively stored over long periods of time without exhibiting significant change in physio-chemical characteristics.

### SUMMARY

Embodiments of the invention may provide a crystalline free acid form of the compound according to Formula II (hereinafter compound 1):

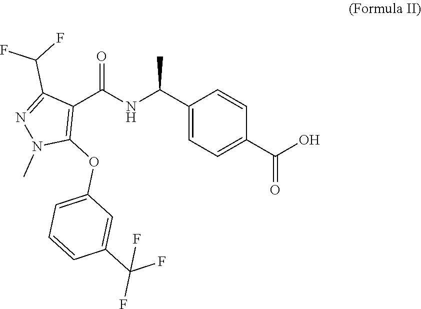 [SMILES: C[C@H](NC(=O)c1c(C(F)F)nn(C)c1Oc1cccc(C(F)(F)F)c1)c1ccc(C(=O)O)cc1]

### (S)-4-(1-(3-(difluoromethyl)-1-methyl-5-(3-(trifluoromethyl)phenoxy)-1H-pyrazole-4-carboxamido)ethyl)benzoic acid

Note that if there is any conflict between the stated IUPAC name of compound 1 or any other compound reported herein and its fully drawn chemical structure, the structure controls.

In some embodiments, the crystalline free acid form of the compound of Formula II gives peaks in an X-ray powder diffraction (XRPD) spectra of at least one, two, three, four, five, six or more of the following value ranges of 20° (±0.2°): 8.2, 10.3, 12.6, 14.2, 14.5, 14.7, 14.8, 15.3, 15.8, 16.4, 17.8, 18.3, 18.4, 19.2, 19.5, 20.4, 23.0, 23.4, 24.2, 25.4, 25.8, 26.3, 26.5, 27.7, 28.9, 29.4, 30.0, 30.4, 31.2, 32.0, 32.6, 33.3, 34.0 and 34.2. In some embodiments, the crystalline free acid form of compound 1 is characterized by an XRPD pattern substantially as indicated in FIG. 1. In some embodiments the crystalline free acid form of the compound gives peaks in X-ray powder diffraction spectra at the following ranges of 2θ° (±0.2°): 8.2, 10.3, 12.6, 14.2, 14.5, 14.7, 14.8, 15.3, 15.8, 16.4, 17.8, 18.3, 18.4, 19.2, 19.5, 20.4, 23.0, 23.4, 24.2, 25.4, 25.8, 26.3, 26.5, 27.7, 28.9, 29.4, 30.0, 30.4, 31.2, 32.0, 32.6, 33.3, 34.0 and 34.2. In other embodiments, the crystalline free acid form of the compound gives peaks in X-ray powder diffraction spectra at the following ranges of 2θ° (±0.2°): 14.7, 14.8, 15.3, 16.4, 18.3, 18.4, 19.5, 20.4, 23.0, 24.2, 27.7, 33.3, and 34.2. In further embodiments, the crystalline free acid form of the compound gives peaks in X-ray powder diffraction spectra at the following ranges of 2θ° (±0.2°): 14.7, 14.8, 18.4, 19.5, 20.4, 23.0, 24.2 and 33.3. In some embodiments, the crystalline free acid form of the compound gives peaks in X-ray powder diffraction spectra at the following ranges of 2θ° (±0.2°): 18.4, 20.4 and 24.2.

The 2θ° values of the crystalline free acid form of the compound according to Formula II were identified through the use of 50 kV, 300 mA X-ray diffractometer, model: RINT TTR-III (Rigaku), using Cu Ku radiation at 50 kV and 300 mA, that was run in a continuous scan mode and a parallel beam measurement mode with a scan speed of 5.00°/min. The detector was a scintillation counter and the tube voltage was 50 kV with a tube current of 300 mA. The step width was 0.020 and an incident slit box of 0.50 mm was used. The diffractometer was also run at a scan range of 5 to 350 at room temperature.

In some embodiments, the crystalline free acid form is characterized by a differential scanning calorimetry (DSC) thermograph substantially the same as shown in FIG. 2. In other embodiments, the crystalline free acid form of compound 1 is characterized by an endothermic peak at 150° C.

Embodiments may provide a pharmaceutical composition comprising a crystalline form of compound 1 as described herein and a pharmaceutically acceptable carrier. In some embodiments, the composition is formulated for oral or parenteral administration.

Embodiments may provide a method of making a crystalline free acid form of compound 1. Such methods may include one or more of the steps of
a) preparing a pyrazole according to reaction scheme A:

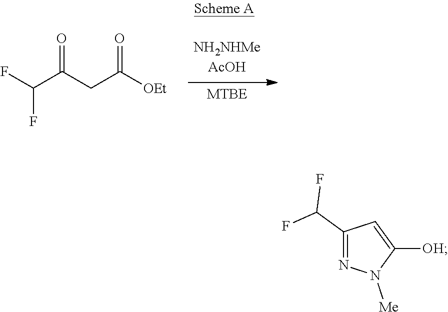 [SMILES: CCOC(=O)CC(=O)C(F)F ; Cn1nc(C(F)F)cc1O]

b) oxidizing the pyrazole according to reaction scheme B:

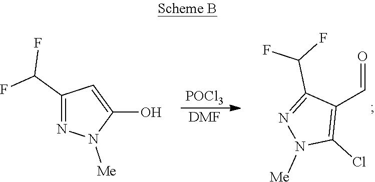

c) functionalizing the oxidized pyrazole according to reaction scheme C:

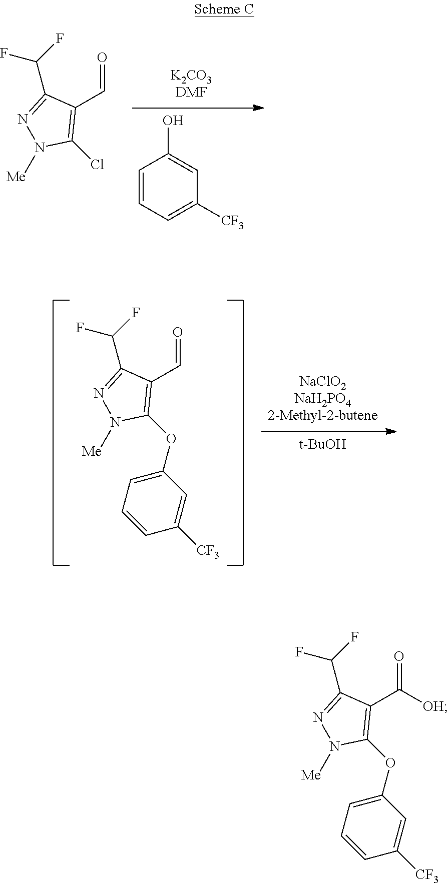

d) coupling the functionalized pyrazole to an amine to form an amide according to reaction scheme D:

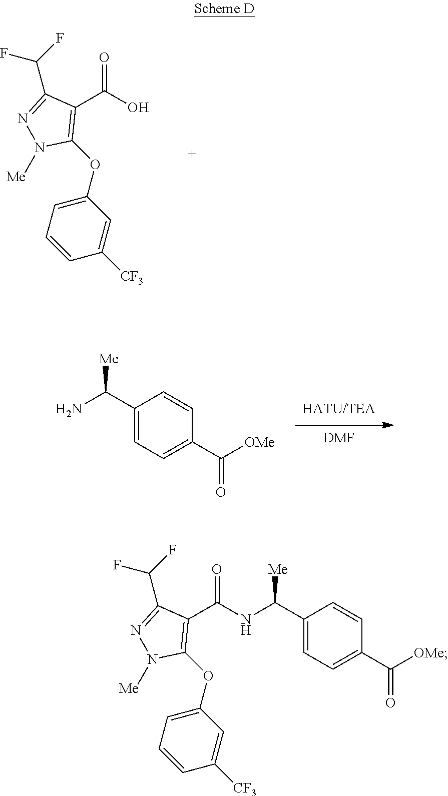

e) preparing the crystalline free acid form of compound 1 according to reaction scheme E:

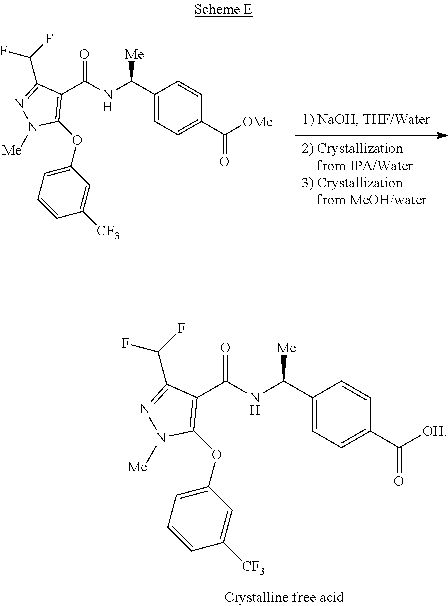

Embodiments may provide a method of treating cancer in a subject in need thereof comprising administering to said subject a treatment effective amount of the crystalline free acid form of compound 1.

Embodiments may provide a method of treating cancer in a subject in need thereof, comprising: detecting an altered EP4 status (e.g., increased expression of EP4) in a biological sample of cells, and if said biological sample possesses cancerous cells with altered EP4 status, administering the crystalline free acid form of compound 1 to said subject in a treatment-effective amount.

Embodiments may provide use of the crystalline free acid form of compound 1 in a method of treatment of cancer.

Embodiments may provide use of the crystalline free acid form of compound 1 in the preparation of a medicament for the treatment of cancer.

### BRIEF DESCRIPTION OF THE FIGURES

FIG. 1 presents an XRPD spectrum obtained from the crystalline free acid form of compound 1.

FIG. 2 present a DSC thermograph of the crystalline free acid form of compound 1.

FIG. 3 presents the hygroscopicity of the crystalline free acid form of compound 1.

### DETAILED DESCRIPTION OF EMBODIMENTS

Provided herein are crystalline forms of the compound 1:

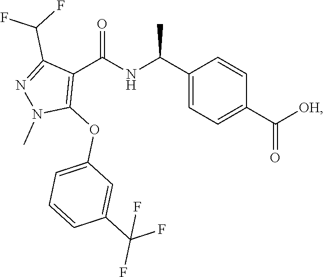 [SMILES: C[C@H](NC(=O)c1c(C(F)F)nn(C)c1Oc1cccc(C(F)(F)F)c1)c1ccc(C(=O)O)cc1]

which are useful as a selective EP4 antagonists.

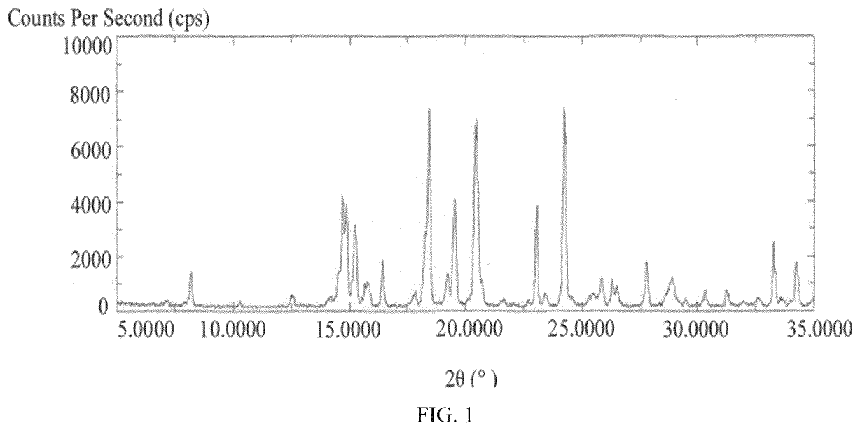

In some embodiments, the crystalline form of compound 1 is the free acid form. An embodiment of the crystalline free acid form of compound 1 gives peaks in an X-ray powder diffraction (XRPD) spectra at one, two, three, four, five, six or more of the following value ranges of 2θ° (±0.2°): 8.2, 10.3, 12.6, 14.2, 14.5, 14.7, 14.8, 15.3, 15.8, 16.4, 17.8, 18.3, 18.4, 19.2, 19.5, 20.4, 23.0, 23.4, 24.2, 25.4, 25.8, 26.3, 26.5, 27.7, 28.9, 29.4, 30.0, 30.4, 31.2, 32.0, 32.6, 33.3, 34.0 and 34.2. For example, the crystalline free acid form of compound 1 may exhibit at least one, two, three, four, five or six values selected from the group consisting of 2θ° (±0.2°): 14.7, 14.8, 15.3, 16.4, 18.3, 18.4, 19.5, 20.4, 23.0, 24.2, 27.7, 33.3 and 34.2. In further embodiments, the crystalline free acid form of the compound gives peaks in X-ray powder diffraction spectra at the following ranges of 2θ° (±0.2°): 14.7, 14.8, 18.4, 19.5, 20.4, 23.0, 24.2 and 33.3. In some embodiments, the crystalline free acid form of the compound gives peaks in X-ray powder diffraction spectra at the following ranges of 2θ° (±0.2°): 18.4, 20.4 and 24.2. In some embodiments, the crystalline free acid form of compound 1 is characterized by an XRPD pattern substantially as shown in FIG. 1.

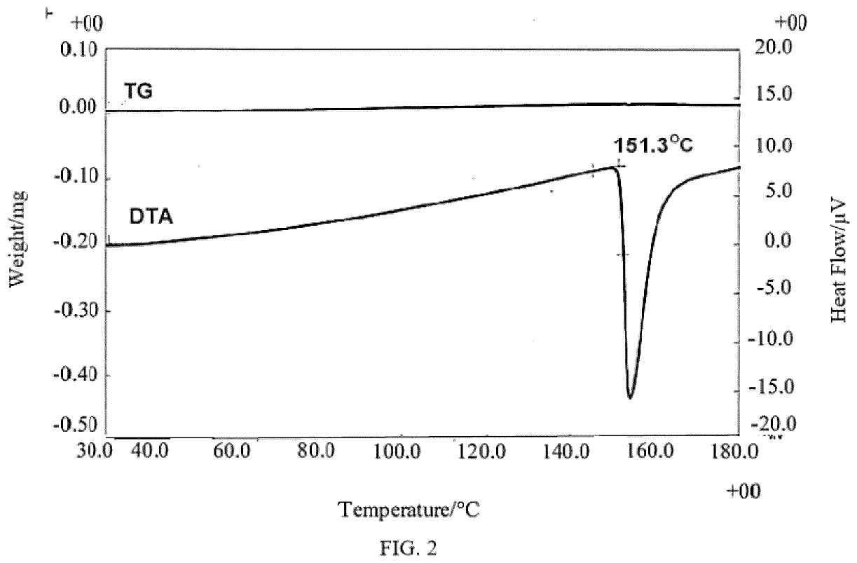

In some embodiments, the crystalline free acid form is characterized by a differential scanning calorimetry (DSC) thermograph substantially the same as shown in FIG. 2.

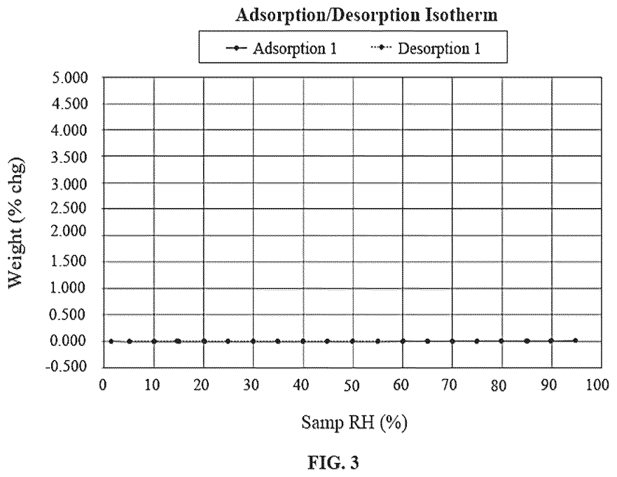

In some embodiments, the crystalline free acid form is characterized by a hygroscopicity substantially the same as shown in FIG. 3.

As used herein, “substantially as shown,” “substantially the same,” or “substantially as indicated” with reference to data such as a spectrum means that the skilled person, when comparing such spectra obtained using the same methods of collection of the data, for example, such as shown in FIG. 1, would conclude that the spectra are similar enough to be indicative of the same crystalline free acid form of the compound as taught herein.

Also provided are methods of synthesizing and crystallizing compounds as taught herein. For a crystalline free acid form, methods may include one or more of the steps of:
a) preparing a pyrazole according to reaction scheme A:

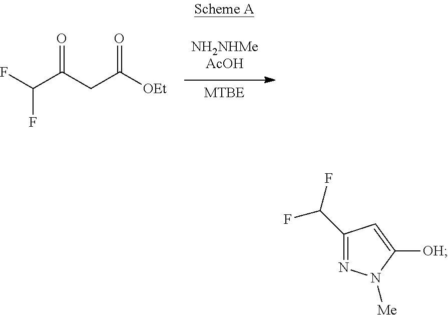 [SMILES: CCOC(=O)CC(=O)C(F)F ; "MTBE" ; Cn1nc(C(F)F)cc1O]

b) oxidizing the pyrazole according to reaction scheme B:

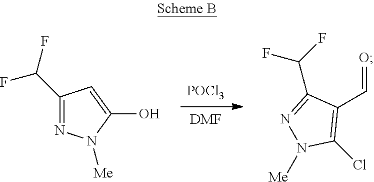

c) functionalizing the oxidized pyrazole according to reaction scheme C

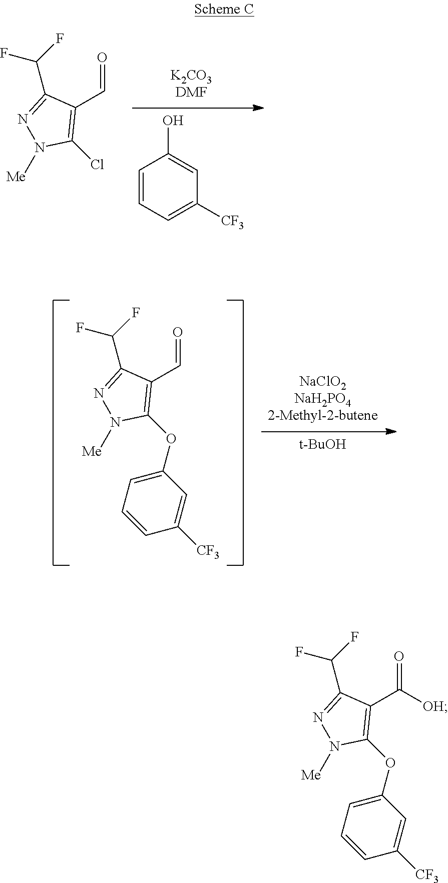

d) coupling the functionalized pyrazole to an amine to form an amide according to reaction scheme D:

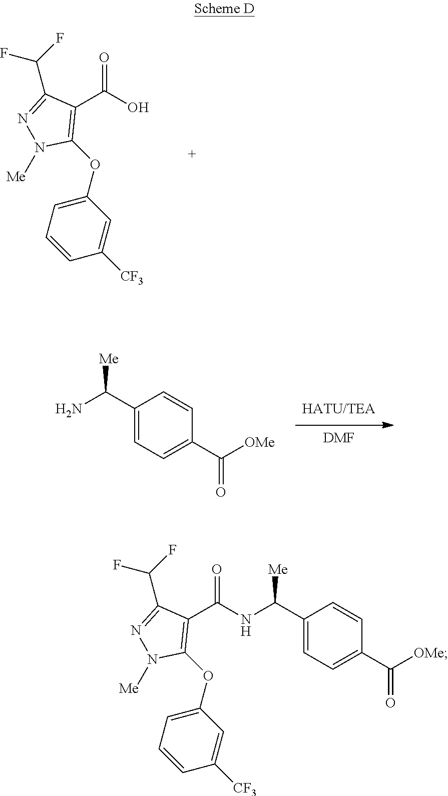

e) creating the crystalline free acid form of compound 1 according to reaction scheme E:

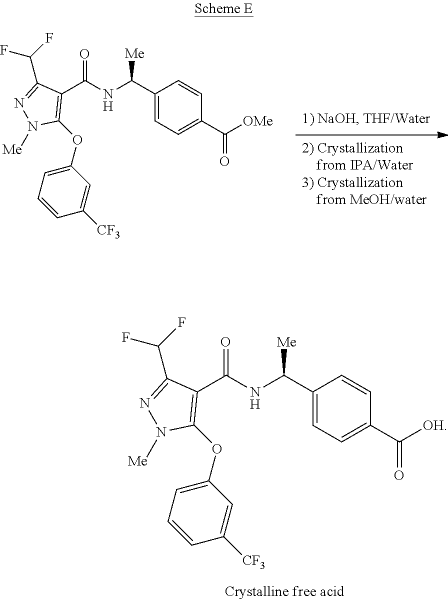

A crystalline compound as reported herein may be combined with a pharmaceutically acceptable carrier to provide pharmaceutical formulations thereof. The particular choice of carrier and formulation will depend upon the particular route of administration for which the composition is intended. In some embodiments, the carrier is selected so as to maintain the crystalline form of the compound prior to administration.

As used in the specification and claims, the singular forms “a,” “an,” and “the” include plural references unless the content dictates otherwise. Thus, for example, reference to “an HCl salt” makes reference to monohydrochloride salts, dihydrochloride salts, 1.5 hydrochloride salts, and other stoichiometric and nonstoichiometric hydrochloride salts.

“Pharmaceutically acceptable carrier” as used herein refers to a nontoxic carrier, adjuvant, or vehicle that does not destroy the pharmacological activity of the compound with which it is formulated. In some embodiments, the pharmaceutically acceptable carrier is selected so as to maintain the crystalline free acid form of the compound. Pharmaceutically acceptable carriers, adjuvants or vehicles may include, but are not limited to, sorbic acid, potassium sorbate, partial glyceride mixtures of saturated vegetable fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene glycol and wool fat.

The compositions of the present invention may be suitable for oral, parenteral, topical or implanted reservoir administration. In some embodiments, the formulation comprises ingredients that are from natural or non-natural sources. In some embodiments, the formulation or carrier may be provided in a sterile form. Non-limiting examples of a sterile carrier include endotoxin-free water or pyrogen-free water.

The term “parenteral” as used herein includes subcutaneous, intravenous, intramuscular, intra-articular, intra-synovial, intrasternal, intrathecal, intrahepatic, intralesional and intracranial injection or infusion techniques. In particular embodiments, the compounds are administered intravenously, orally, subcutaneously, or via intramuscular administration. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids and their glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents that are commonly used in the manufacture of pharmaceutically acceptable solid, liquid, or other dosage forms may also be used for the purposes of formulation.

For oral administration, a compound or salt may be provided in an acceptable oral dosage form, including, but not limited to, capsules, tablets, aqueous suspensions or solutions. In the case of tablets for oral use, carriers commonly used include lactose and corn starch. Lubricating agents, such as magnesium stearate, may also be added. For oral administration in a capsule form, useful diluents include lactose and dried cornstarch. When aqueous suspensions are required for oral use, the active ingredient may be combined with emulsifying and suspending agents. If desired, certain sweetening, flavoring or coloring agents may also be added. In addition, preservatives may also be added. Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

In some embodiments, the crystalline free acid form of compound 1 is formulated into a capsule, wherein 5 mgs to 50 mgs, 10 mgs to 40 mgs, or 20 mgs to 30 mgs of compound 1 is present within the capsule.

In other embodiments, the capsule comprises a capsule shell made of hypromellose.

In some embodiments, the crystalline free acid form of compound 1 is formulated into a tablet, wherein compound 1 undergoes a wet-granulation process during tablet formation.

In other embodiments, the tablet comprises lactose monohydrate, low-substituted hydroxypropyl cellulose, hydroxypropyl cellulose, microcrystalline cellulose, magnesium stearate and/or water.

The tablet may have a size of between 1 mm to 7 mm, 2 mm to 7 mm, 4 mm to 6.5 mm or 5.5 mm to 6.5 mm. In some embodiments, the tablet has a size of 6.5 mm.

### Subjects and Methods of Use

The crystalline form of the compound as taught herein may be used to treat a cancer such as skin cancer, breast cancer, colorectal cancer, prostate cancer, kidney cancer, cervical cancer, ovarian cancer, endometrial cancer, glioblastoma, head and neck cancer, medulloblastoma, lung cancer, or urinary tract cancers.

“Treatment,” “treat,” and “treating” refer to reversing, alleviating, delaying the onset of, inhibiting the progress of, or otherwise ameliorating a disease or disorder as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

“Patient” or “subject”, as used herein, means an animal subject, preferably a mammalian subject, and particularly human subjects (including both male and female subjects, and including neonatal, infant, juvenile, adolescent, adult and geriatric subjects). Subjects may also include other mammalian subjects (e.g., dog, cat, horse, cow, sheep, goat, monkey, bird, etc.), for laboratory or veterinary purposes.

In some embodiments, treatment is provided to a subject having a cancer with altered EP4 status.

In some embodiments, treatment may include or be performed in conjunction with analyzing (e.g., measuring or assaying for) EP4 status in a biological sample containing cells of said cancer, and if said cancer exhibits an EP4 alteration, treating a subject with a treatment effective amount of an active agent as described herein.

“Altered status” as used herein with reference to EP4 includes an increased expression thereof (e.g., increased levels of the mRNA or increased levels of the protein), increased copy number in the genome, and/or increased activity of the encoded protein as a result of mutation, etc., as compared to a corresponding non-cancerous tissue. In some embodiments, altered status of EP4 includes gene and/or encoded protein mutations that result in an increase in activity or are otherwise associated with a more aggressive form of hepatocellular carcinoma.

“Expression” of EP4 means that a gene encoding the same is transcribed, and preferably, translated. Typically, expression of a coding region will result in production of the encoded polypeptide.

The EP4 protein is known, and its altered status and/or expression may be measured using techniques standard in the art, e.g., genomic analysis of mutations or copy number aberrations such as by nucleic acid amplification, sequencing analysis, and/or hybridization-based techniques, RNA expression analysis such as northern blot or qRT-PCR, western blot or other immunoblot or immunoassay, fluorescent activated cell sorting (FACS), etc.

In order that the invention described herein may be more fully understood, the following examples are set forth. It should be understood that these examples are for illustrative purposes only and are not to be construed as limiting.

### EXAMPLES

Definitions: The following abbreviations have the indicated meanings:
ACN: Acetonitrile
DCM: Dichloromethane
DMF: Dimethylformamide
K₂CO₃: Potassium carbonate
MeOH: Methanol
EtOAc: ethyl acetate
prep-HPLC: Preparative high-performance liquid chromatography
TEA: Triethylamine
THF: Tetrahydrofuran
MTBE: Methyl tert-butyl ether
AcOH: Acetic acid
POCl₃: Phosphoryl chloride
t-BuOH: tert-butanol
IPA: Isopropanol
V: volumetric equivalent

### Example 1: Procedures for the Synthesis of Free Acid Crystalline Form of Compound 1

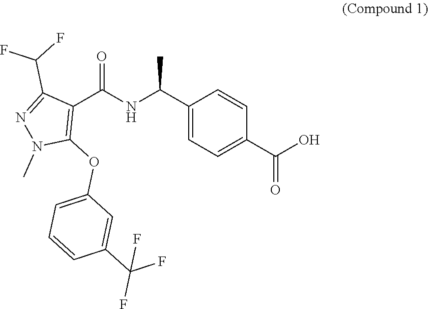 [SMILES: C[C@H](NC(=O)c1c(C(F)F)nn(C)c1Oc1cccc(C(F)(F)F)c1)c1ccc(C(=O)O)cc1]

### Example 1A

### General Synthetic Scheme I:

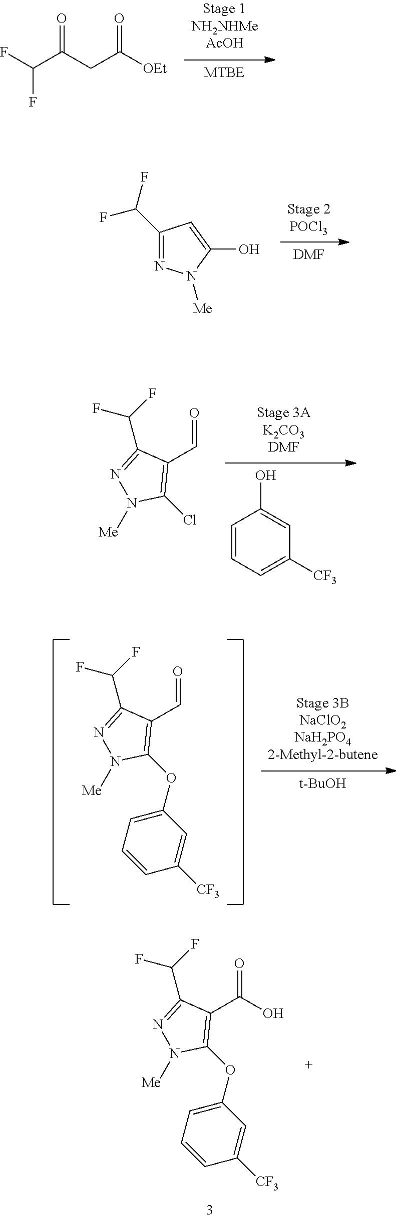

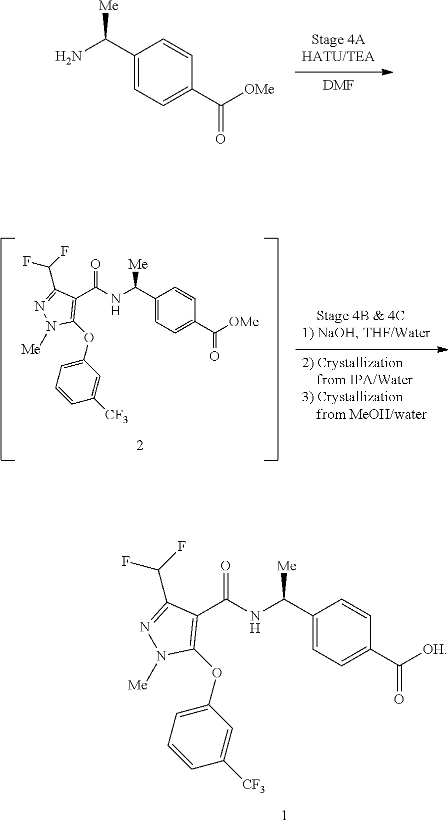

Compounds 21 to 4 can be prepared as described in U.S. Pat. No. 8,686,018 which is wholly incorporated by reference herein.

### Step 1.1 (Stage 4A)

Compound 3 (1.0 kg) and 5 L of DMF were charged into the reactor at 20-30° C. and then the reaction mass was cooled to −5° C. to 10° C. Compound 4 (0.56 kg) and HBTU (1.36 kg) were added sequentially into the reactor at −5 to 10° C. Triethylamine (TEA) (0.6019 kg) was slowly added at −5 to 10° C. The reaction mass was stirred at 0-10° C. for 1 to 3 hrs. The progress of the reaction was monitored by HPLC.

Sampling procedure: Take 2 ml of reaction mass and give for HPLC.

Acceptance criteria: Compound 3 is no more than (NMT) 1.0%.

If reaction not completed: stir the reaction mass further at 0-10° C. for 1-3 hrs and check the content of Compound 3 as per the acceptance criteria.

Once completed, 4 L of water was slowly charged into the reaction mass at 0-20° C. and the reaction mass was extracted with 10 L of ethyl acetate and again with 6 L of ethyl acetate (Note: if the separation of the layers doesn't occur during first extraction, then rise the temperature to 10-20° C., add 2 L of ethyl acetate, stir for 10-30 min, settle and separate the ethyl acetate layer). The combined ethyl acetate layer was washed sequentially with an 8% NaHCO3 solution (water 5 L+NaHCO₃ 0.4 Kg) and with 10% Sodium chloride solution (water 5 L+Sodium chloride 0.5 Kg) at 20-30° C. The ethyl acetate layer was distilled at below 55° C. under vacuum to yield Compound 2 (yield=1.48 kg w/w w.r.t Compound 3, assume 100% yield) and used in Step 2.1 without isolation.

### Chirality Control:

The chirality of Compound 2 is obtained commercially from the (S)-methyl 4-(1-aminoethyl)benzoate (Compound 4) starting material.

### Step 2.1 (Stage 4B)

THF (2.96 L) was charged into a reactor with Compound 2 (crude material, 1.48 kg) at a temperature below 55° C. THF was distilled out of the reactor under vacuum and the reactor was again charged with THF (2.96 L) at a temperature below 55° C. THF was again distilled out of the reactor under vacuum and the reactor was charged again with THF (7.4 L). The reaction mass was cooled to 10 to 30° C. NaOH (0.3 kg in 7.4 L water) was charged into the reaction mass at 10-30° C. The temperature was increased to 35 to 50° C. and the reaction mass was stirred for 8-14 hr. Progress of the reaction was monitored by HPLC.

Sampling procedure: Take 2 ml of reaction mass for HPLC.

Acceptance criteria: Compound 2 is not more than 1.0%

If reaction not completed: stir the reaction mass further at 35-50° C. for 1-3 hrs. Once completed, the reaction mass was cooled to a temperature of 0-10° C. The pH of the reaction mass was adjusted to 1.5 to 2.5 by slowly adding 10 L HCl solution (1.48 L Con.HCl in 13.32 L water) to the reaction mass at 0-30° C. The reaction mass was extracted with ethyl acetate (11.84 L) and again with ethyl acetate (10.36 L). The combined ethyl acetate layer were washed with a 10% Sodium chloride solution (water 7.4 L+Sodium chloride 0.74 Kg). The organic layer was transferred into another reactor through particle free filtration. The ethyl acetate was distilled out completely at a temperature below 55° C. under vacuum. IPA (1.48 L) was added to the residue at a temperature below 55° C. The IPA was distilled out of the reaction mass under vacuum. IPA (7.4 L) was again charged into the reaction mass and the reaction mass was heated to a temperature of 55-65° C. to get a clear solution. The reaction mass was stirred at 55-65° C. for 10-30 minutes and then cooled to 40-50° C. Water (10.36 L) was slowly added to the reaction mass to reaction mass at 40-50° C. and was stirred the reaction mass for 30-60 minutes at 40-50° C. The reaction mass was cooled to 20 to 30° C. and stirred for 60±15 min. at 20 to 30° C. The reaction mass was further cooled to 5 to 15° C. and stirred for 2-4 hr at 5 to 15° C. The precipitated solid was then filtered and the solid was washed sequentially with 2.96 L of (1:2 V/V) IPA+water mixture and then with 1.48 L water followed by with 1.48 L of (1:2 v/v IPA+water mixture. The wet material was dried at 55-65° C. for 10-20 hr. The progress of drying of the material was monitored by water content using Karl fischer titration. Acceptance criteria of the water content is not more than 1.0%. If the water content does not meet the acceptance criteria, then continue drying for 2-4 hours at 55-65° C. and check the water content. After meeting the target criteria for the drying, unload the material from the drier to yield the crude title compound 1 (0.9-1.3 kg, yield=70-90%) (Stage 4B).

### Step 3.1 (Stage 4C)

Methanol (5 L) was charged into a reactor at 20-30° C. The product from crude compound 1 Stage 4B (1.0 kg) was added to the reactor. The reaction mass was heated to 55-65° C. and stirred for 30-45 minutes to get a clear solution. The reaction mass was then cooled to 30-50° C. and transferred into another reactor through particle free filtration (5 Micron filter). The reaction mass was rinsed with methanol (2 L) at 30-50° C. and transferred into another reactor through particle free filtration (5 Micron filter). The reaction mass was cooled to 5-15° C. and water (13 L) was slowly added into the reaction mass at 5-25° C. After completion of the water addition, the reaction mass was heated to a temperature of 20-30° C. and stirred at the same temperature for 2-4 hr. The precipitated solid was filtered and the solid was washed with 3 L of (1:2 V/V) MeOH+water mixture. The wet material was dried at 55-65° C. for 10-20 hr.

The progress of drying of the material was monitored by (i) water content using Karl Fischer titration and (ii) methanol content by GC. Acceptance criteria of the water content is not more than 1.0% and the acceptance criteria of methanol content is 3000 ppm. If the water content and methanol content do not meet the acceptance criteria, then continue drying for 2-4 hours at 55-65° C. and check the water content and methanol content. After meeting the target criteria for the drying, unload the material from the drier to yield the crystalline free acid form of Compound 1 as a white solid (0.9-0.98 kg, yield=90-98%).

### Example 1B

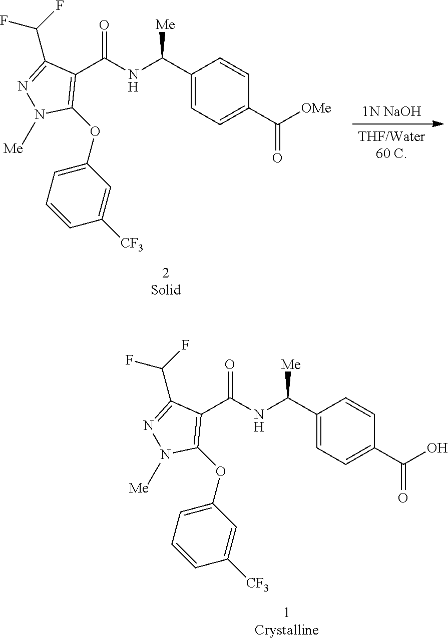 [SMILES: COC(=O)c1ccc([C@H](C)NC(=O)c2c(C(F)F)nn(C)c2Oc2cccc(C(F)(F)F)c2)cc1 ; "1N NaOH" ; "THF/Water60 C." ; C[C@H](NC(=O)c1c(C(F)F)nn(C)c1Oc1cccc(C(F)(F)F)c1)c1ccc(C(=O)O)cc1]

Alternatively, Compound 1 can be produced from the following synthesis. Compound 2 can be prepared as described in U.S. Pat. No. 8,686,018, which is wholly incorporated by reference herein.

A reactor was charged with Compound 2 (1 eq) followed by THF (5 V). The reaction was stirred and 1M NaOH (5 V) was added at 20-25° C. The reaction was heated to 60-65° C. and monitored for completion. (˜4-6 h). Upon completion, the reaction was cooled to 0-10° C. and charged slowly with 1M HCl (6 V). The reaction was then charged with EtOAc (8 V), stirred, phase separated and then the organic phase was removed from the aqueous phase. The aqueous phase was back extracted with EtOAc (8V). The organic phases were then combined and washed with 12% brine (5V). The aqueous phase was removed and the organic phase was concentrated down. The concentrated organic phase was then azeotroped with MeOH twice (1V). The crude organic material was then charged with MeOH (2 V) and heated to 50-60° C. until the solution became clear. Water (6V) was then slowly added to the solution. The solution was cooled at a rate of 0.1° C./min to a temperature of 15-20° C. The precipitated solids were then filtered out of the solution and washed twice with 1 V of MeOH:Water (1:3 v/v). The collected solids were dried until a constant weight was achieved, overall yielding Compound 1 (0.85 W, 87%) as a white solid.

### Example 2: XRPD Characterization of Crystalline Free Acid Form of Compound 1

The X-ray powder diffraction (XRPD) pattern for crystalline free acid form of Compound 1 is shown in FIG. 1.

### Example 3: TG-DTA Characterization of Crystalline Free Acid Form of Compound 1

Thermogravimetry and differential thermal analysis (TG-DTA) of crystalline free acid form of Compound 1 is shown in FIG. 2. In the DTA curve, an endothermic peak was observed at around 150° C. (as an onset). In the TG curve, no measurable weight loss was observed up to 180° C. The melting range of crystalline free acid form of Compound 1 was observed from 152 to 154° C. in accordance with USP-NF 37.

### Example 4: Hygroscopicity Characterization of Crystalline Free Acid Form of Compound 1

Hygroscopicity of crystalline free acid form of Compound 1 was measured using the dynamic vapor sorption (DVS) method. The adsorption and desorption isotherm for crystalline free acid form of Compound 1 is shown in FIG. 3. No significant weight change of crystalline free acid form of Compound 1 was observed over a relative humidity range from 5% to 95% RH at 25° C. The results indicated that crystalline free acid form of Compound 1 was non-hygroscopic.

### Summary Data of Crystalline Free Acid Form of Compound 1:

The physical and chemical properties of crystalline free acid of Compound 1 are summarized in Table 1.

TABLE 1
Physical and chemical properties of
crystalline free acid of Compound 1

| Test | Property |
|---|---|
| Appearance | White solid |
| Dissociation | 4.1 |
| Constant (pKa)^{a} | |
| Melting Range | From 152 to 154° C. |
| Thermal Analysis | A single endothermic peak observed at around 150° C. |
| | (onset) |
| Solubility in | Freely soluble in dimethyl sulfoxide, methanol, and |
| Organic Solvents | acetone, soluble in acetonitrile, sparingly soluble in |
| and Water | ethanol and ethyl acetate, slightly soluble in 2- |
| | propanol, very slightly soluble in diethyl ether and |
| | practically insoluble in water and heptane. |
| Solubility in | Very slightly soluble in Britton-Robinson buffer (I = |
| Aqueous | 0.3, pH 9) and practically insoluble in 0.1N HCl and |
| Solutions | Britton-Robinson buffer (I = 0.3, pH 3, 5, 7). |
| Hygroscopicity | Non-hygroscopic. |
| Crystal Form | Polymorphism has not been observed for E7046 drug |
| | substance. |

I = ionic strength
^{a}Determined by capillary electrophoresis (CE) using buffer solution (I = 0.05, 25° C.).

1. A crystalline form of compound 1:

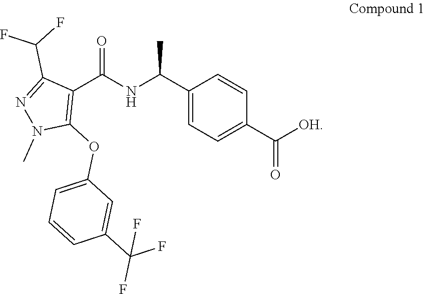 [SMILES: C[C@H](NC(=O)c1c(C(F)F)nn(C)c1Oc1cccc(C(F)(F)F)c1)c1ccc(C(=O)O)cc1]

2. The crystalline compound of claim 1, wherein said compound is a crystalline free acid form of compound 1.

3. The crystalline free acid compound of claim 2, wherein the crystalline free acid form compound exhibits at least the following X-ray powder diffraction peaks, 2θ° (±0.2°): 18.4, 20.4, and 24.2.

4. The crystalline free acid compound of claim 3, wherein the crystalline free acid form compound exhibits at least the following X-ray powder diffraction peaks, 2θ° (±0.2°): 14.7, 14.8, 18.4, 19.5, 20.4, 23.0, 24.2, and 33.3.

5. The crystalline free acid compound of claim 4, wherein the crystalline free acid form compound exhibits at least the following X-ray powder diffraction peaks, 2θ° (±0.2°): 14.7, 14.8, 15.3, 16.4, 18.3, 18.4, 19.5, 20.4, 23.0, 24.2, 27.7, 33.3, and 34.2.

6. The crystalline free acid compound of claim 5, wherein the crystalline free acid form compound exhibits at least the following X-ray powder diffraction peaks, 2θ° (±0.2°): 8.2, 10.3, 12.6, 14.2, 14.5, 14.7, 14.8, 15.3, 15.8, 16.4, 17.8, 18.3, 18.4, 19.2, 19.5, 20.4, 23.0, 23.4, 24.2, 25.4, 25.8, 26.3, 26.5, 27.7, 28.9, 29.4, 30.0, 30.4, 31.2, 32.0, 32.6, 33.3, 34.0 and 34.2.

7. The crystalline free acid compound of claim 2, wherein the crystalline free acid form is characterized by an X-ray powder diffraction (XRPD) pattern substantially as shown in FIG. 1.

8. The crystalline free acid compound of claim 2, wherein the crystalline free acid form is characterized by a differential scanning calorimetry (DSC) thermograph substantially the same as shown in FIG. 2.

9. The crystalline free acid compound of claim 2, wherein the crystalline free acid form is characterized by a hygroscopicity substantially the same as shown in FIG. 3.

10. A pharmaceutical composition comprising the crystalline compound of claim 1 and a pharmaceutically acceptable carrier.

11. The pharmaceutical composition of claim 10, wherein said composition is formulated for oral administration.

12. A method for preparing the crystalline free acid form compound of claim 2, comprising any one or more of the following steps:
a) creating a pyrazole according to reaction scheme A: 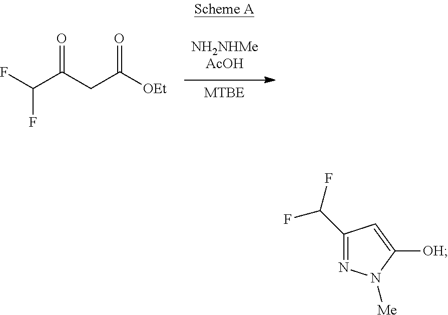 [SMILES: CCOC(=O)CC(=O)C(F)F ; "MTBE" ; Cn1nc(C(F)F)cc1O]
b) oxidizing the pyrazole according to reaction scheme B: 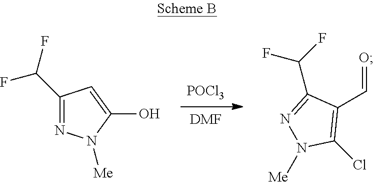
c) functionalizing the oxidized pyrazole according to reaction scheme C: 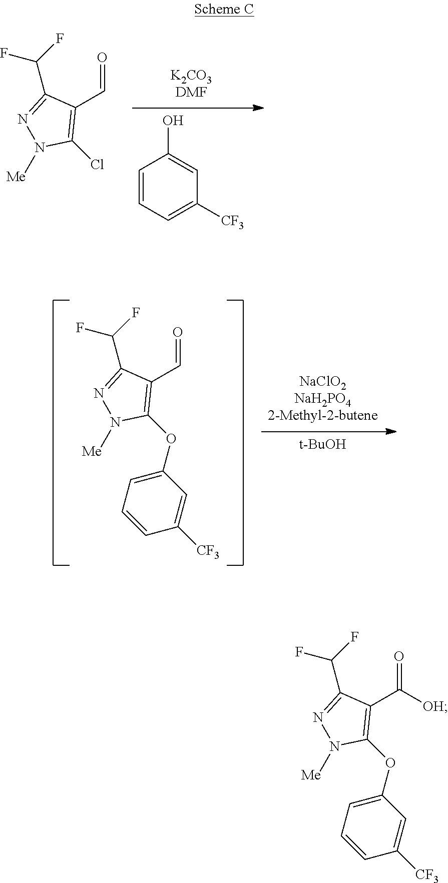
d) coupling the functionalized pyrazole to an amine to form an amide according to reaction scheme D: 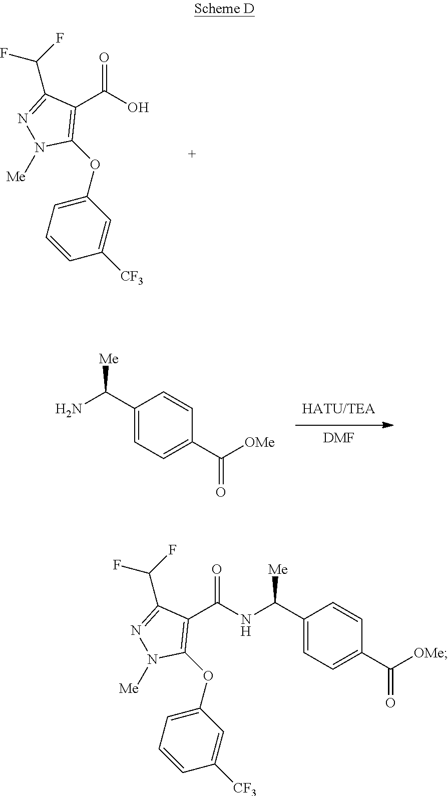
e) creating the crystalline free acid form of compound 1 according to reaction scheme E:

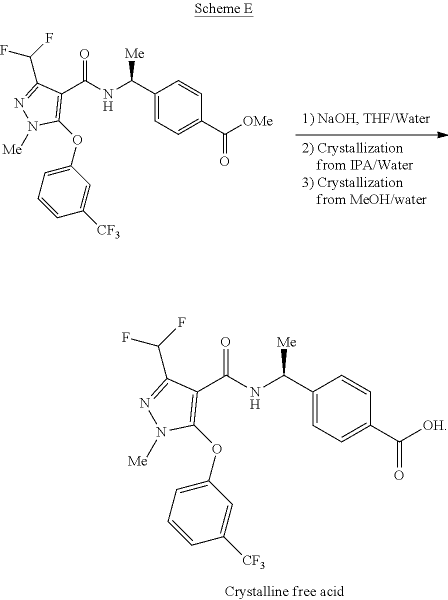

13. A method of treating cancer in a subject in need thereof comprising administering to said subject a treatment effective amount of the compound of claim 1.

14. The method of claim 13, wherein the cancer is skin cancer, breast cancer, colorectal cancer, prostate cancer, kidney cancer, cervical cancer, ovarian cancer, endometrial cancer, glioblastoma, head and neck cancer, medulloblastoma, lung cancer, or urinary tract cancer.

15. The method of claim 13, wherein said cancer has altered EP4 status.

16. The method of claim 15, wherein said altered EP4 status comprises increased expression of EP4.

17. A method of treating cancer in a subject in need thereof, comprising:
detecting an altered EP4 status in a biological sample containing cells of said cancer, and if said cancer has said altered EP4 status,
administering a crystalline form of the compound of claim 1 to said subject in a treatment-effective amount.

18. The method of claim 17, wherein said altered EP4 status comprises increased expression of EP4.

19.-25. (canceled)
